(19) United States
(12) Patent Application Publication
(10) Pub. No.: US 2008/0192483 A1
(43) Pub. Date: Aug. 14, 2008

(54) In-Ceiling Focus Located Surgical Lighting

(76) Inventor: John R. Mangiardi, Greenwich, CT (US)

Correspondence Address: HUNTON & WILLIAMS/NEW YORK;INTELLECTUAL PROPERTY DEPT., 1900 K STREET, N.W., SUITE 1200, WASHINGTON, DC, 20006-1109, US

(21) Appl. No.: 11/996,018

(22) PCT Filed: Jul. 20, 2006
(86) PCT No.: PCT/US06/28225

§ 371 Date: Feb. 6, 2008

Related U.S. Application Data
(60) Provisional application No. 60/701,106, filed on Jul. 20, 2005.

(51) Int. Cl. F21S 8/02

(52) U.S. Cl. 362/319

(57) ABSTRACT

An invention that provides improved means for automatic illumination of a surgical site is disclosed. By way of utilizing a wireless radio-frequency transmitter adapted to transmit a distance signal to a receiver, which provides adjustment, by way of an actuator, of a single light source or an array of light sources, an illuminating field is provided with the further advantage that shadows are minimized. In addition, the transmitter, in one embodiment, is of a small profile and constructed in biocompatible materials. The device may be either sterilizable for reuse or disposable, thereby allowing packing of the transmitter in a sterile container thereby further allowing placement of the device in, on, or by a surgical site. The present invention also discloses an array of lights that are arrayed more distant from each other than typical and yet provides a focused, intense illumination field with maximal line of sight approach owing to the discrete (recessed) housing of the light sources. Lastly, the recessed housing allows more efficient use of overhead working space.

### CROSS-REFERENCE TO RELATED APPLICATIONS

This application claims the benefit of provisional patent application U.S. Ser. No. 60/701,106, filed Jul. 20, 2005 by the present inventor. The contents of U.S. Ser No. 60/701,106 are expressly incorporated herein by reference thereto.

The following references are hereby explicitly incorporated by reference thereto:
U.S. Pat. No. 4,025,777 (to which German Patent 25 19 426 corresponds)
U.S. Pat. No. 4,639,838
U.S. Pat. No. 4,884,008
U.S. Pat. No. 4,887,196
U.S. Pat. No. 5,347,431
U.S. Pat. No. 5,584,568
German Patent Disclosure Document DE-OS 32 27 494
Applications filed along with present application by current inventor on this date entitled:
   HOSPITAL OPERATING ROOM RE-DESIGN
   AMBIENT LIGHTING IN HOSPITAL SURGICAL ENVIRONMENTS
   USE OF ULTRAVIOLET GERMICIDAL IRRADIATION IN HEALTH CARE ENVIRONMENTS
   IN-WALL WASTE RECEPTACLES FOR HOSPITAL AND LABORATORY ENVIRONMENTS
   MULTIFUNCTIONAL FLOOR PODS
   RE-DESIGN OF OPERATING ROOM TABLES
   ROBOTIC FLOOR CLEANING WITH STERILE, DISPOSABLE CARTRIDGES

### BACKGROUND OF THE INVENTION

1. Field of Invention

The present invention relates to an operating room surgical light, a method of using an operating room light, and more particularly to using an operating room light with auto-adjustable lamp elements therein, so that the optical axes of the light emitted therefrom are directed to a point chosen by a wireless radio-frequency locator.

2. Background of the Invention

Hospital operating rooms typically use a number of lamps arranged about a focal point to illuminate a desired region, usually a surgical site. Because the lights are arrayed about this point, they may be adjusted to provide lighting which does not cast shadows, which would obstruct the view of said surgical site. Typical surgical lighting is arranged by either ceiling or floor mounted devices, which have handles thereon to allow manual adjustment of the lighting. Some devices are positioned manually while others are positioned under powered means such as by electric motors.

U.S. Pat. No. 4,025,777 shows an operating room lamp for placement on a ceiling in an operating room. The lower portion of the device, which carries the light emitting surface, is subdivided to provide room for a plurality of light radiating lamps. The light radiators are located so as to be adjustable. Thus, a lamp guiding arrangement, coupled to the lamps, can change the light axes of the operating room lamp without moving the overall unit itself, so that the light beams from the respective lamp units coverage at a point which can be changed horizontally or vertically. The converging point can be previously determined. The individual lamp units are retained in a housing in a gimbal suspension and interconnected by guide rods and springs. The intersecting point of the three spatial axes can thus be shifted by appropriate readjustment of the control system that is formed by the respective guide rods. A carriage is provided which can be shifted along guide rails. The change or shift can be carried out by drive motors.

German Patent Disclosure Document DE-OS 32 27 494 describes an operating room light specifically adapted for dental work and jaw surgery. A light beam remains continuously directed to the oral region of the patient by automatic tracking of a lamp if the patient's chair is moved. The necessary tracking arrangement includes an ultrasonic transmitter located in the region of the head of the patient, and an ultrasound receiver located in the treatment room, as well as a tracking or targeting circuit. Servo motors or stepping motors are provided to ensure tracking, by bringing the lamp holder in predetermined positions, or inclinations, respectively. Such an operating room light cannot be used for general surgery since the ultrasound transmitter must be located in the immediate vicinity of the operating field to be illuminated, that is, in the region of an open wound. For general surgical purposes, such a system cannot be used since an ultrasound transmitter cannot be placed in an open wound. Errors in adjustment as well as difficulties in handling and sterilization impede such application.

U.S. Pat. No. 4,884,008 shows an operating room light in which the light beams can be automatically adjusted to compensate for movement or change in distance between the operating room light and the operation field, so that any illumination pattern or zone originally set will be retained in its base position. An ultrasonic distance sensor is located on the housing, and facing the operating surface, generates an electrical actual distance signal representative of the actual distance between the housing and the surface. The distance signal is coupled to a servo control circuit, which controls a lamp-adjustment element arrangement. The light beams are emitted from the operating room lamp in a group of beams located in ring shape about the circumference of a circular unit.

In light of the prior art, an invention which can provide on-the-fly tracking, such as the ultrasonic locator in the above-referenced German Disclosure Document, would be of benefit provided it also was adapted for placement near or on a surgical site. In addition, an invention as described that utilized optimal lighting conditions, namely, a light-source emitting very focused and high-intensity light, would be of further benefit. Lastly, the invention as described which also provides a light source from a recessed cavity would allow the location of other overhead equipment, such as a surgical imaging C-arm, and would be of benefit.

### SUMMARY OF THE INVENTION

In keeping with the present invention, it is an object of the invention to provide rapid, on-the-fly tracking of a surgical site or other site to be illuminated.

As such, the present invention discloses the use of a wireless radio-frequency transmitter, such as a Bluetooth transmitter, which is adapted to transmit a distance signal to a receiver. The receiver then provides adjustment by way of an actuator of a single light source or an array of light sources, thereby providing an illuminating field with the further advantage that the latter array design will minimize shadows. In addition, the transmitter, in one embodiment, is of a small profile and constructed in biocompatible materials. The device may be either sterilizable for reuse or disposable, thereby allowing packing of the transmitter in a sterile container and further allowing placement of the device in, on, or by a surgical site.

It is another object of the invention to provide an illumination field that provides maximal shadow cancellation while also providing an intense, focused illumination field.

Light sources congregated about a single point, such as in a circle, allow the individual light sources to be targeted at a particular point. As the radius between any individual light source and the center of the circle decreases, the ability of the light sources to work to cancel shadows decreases. When the radius approaches zero, the resultant array of lights is equivalent to a single beam. As such, the present invention provides an array of lights that have larger than normal radii (radii are with respect to the distance between a light source and the point of illumination) owing to the improvement that individual lights are placed in discrete housing. Unfortunately, as the distance between a light source and a target source increases, the focus and intensity of a beam at the target source decreases. The present invention discloses a means for providing an intense, focused beam with maximal line of sight owing to the discrete housing of the light sources. Because the light sources are housed separately from one another, lamps that are more powerful and other more powerful light-emitting devices may be used, as the generation of localized heat is no longer as great an impediment to the use of very intense, focused light. In other words, the discrete housing minimizes heat build up which would be found if multiple lamps are housed together. It is generally sufficient that the lamps cool by natural radiation of heat, although it is conceived that the lamp housing and supports be composed of materials with high heat dissipation constants such as aluminum.

It is yet another object of the invention to provide an illumination apparatus that integrates with the operating room, such that any light source and its respective housing is recessed into the wall, thereby providing improved space efficiency and the ability to locate additional ceiling mounted equipment near the surgical operating site.

### BRIEF DESCRIPTION OF THE DRAWINGS

The present invention can best be understood in connection with the accompanying drawings. It is noted that the invention is not limited to the precise embodiments shown in drawings, in which:

FIG. 1 is a perspective view of one embodiment of the invention in which a number of recessed lights are arrayed about a central point and focused on said point;

FIG. 2 is a schematic view of prior art, demonstrating the housing of a number of lights focused onto a point;

FIG. 3 is a side-view schematic of the prior art;

FIG. 4 is a schematic representation of two lights focused generally on a point and thereby providing an illumination field;

FIG. 5 is a schematic representation of the lights in FIG. 4 focused on the same point in FIG. 4, said point now more distant from the illumination source;

FIG. 6 is a schematic representation of the target site illuminated in FIGS. 4 and 5, with said lights now more distant from each other;

FIG. 7 is an isometric schematic representation of an example of the present invention.

### BRIEF DESCRIPTION OF REFERENCE NUMERALS

100 Surgical Light; 102 Illumination Target; 104 Operating Table; 110 Prior Art (“PA”) Operating Room Illumination Device; 112 Surgical Light; 114 PA Surgical Lights Housing; 116 PA Ultrasonic Locator; 118 Light Beam; 120 Illumination Field/Target; 122 Ceiling; 124 Table; 150 Control Box; 152 Power; 154 Housing; 156 Lamp; 158 Table; 160 Illumination Target and Locator; 162 Wireless Location Signal; 164 Focused Light Beam; 166 Cable to Control Box; 168 Actuator, Swivel, and Mounting; 172 Cable to Power; 176 Lamp Housing; 178 Lamp Mounting and Swivel

### DETAILED DESCRIPTION OF THE INVENTION

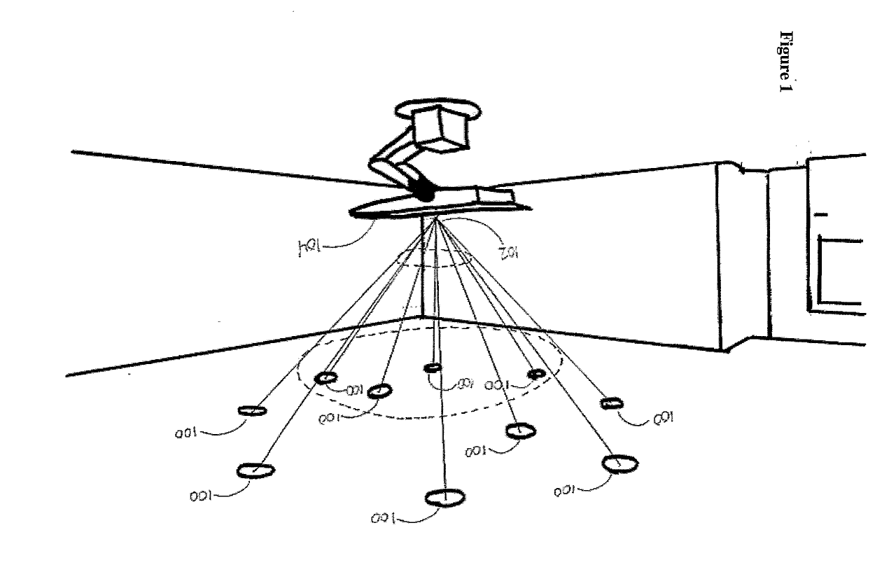

With respect to the drawings, FIG. 1 shows a typical surgical operating room with one possible arrangement of lights 100 positioned in accord with present invention. The dotted circles and lines intersecting through them to a point 102 on surgical table 104 indicates the diminishing illuminating field of the focused lights 100. Since the lights 100 are spaced distant from each other, they are thereby adapted to provide additional lines-of-sight to a target area such as point 102.

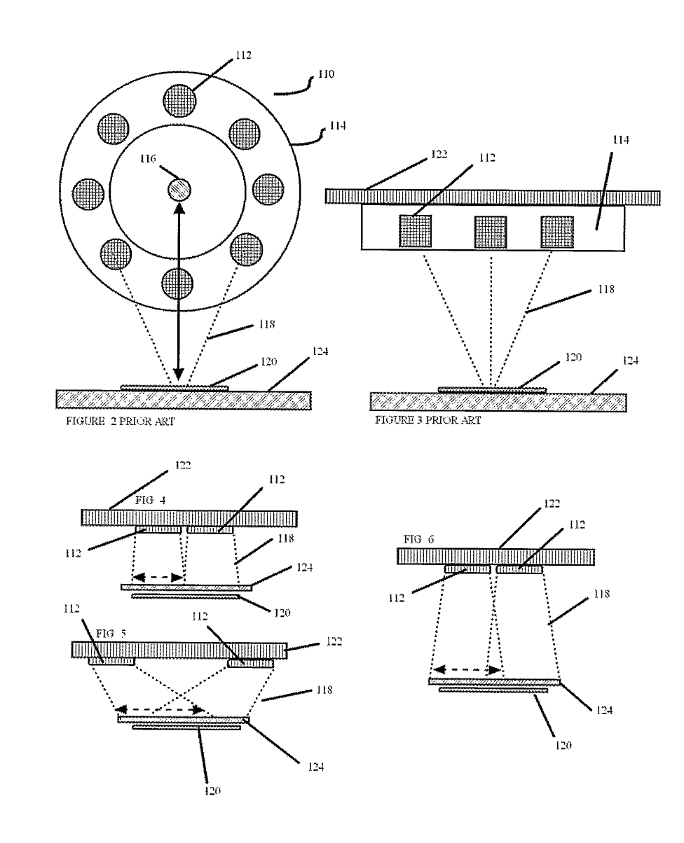

In contrast, in a typical example of the prior art, shown in FIGS. 2 and 3, auto-adjustable surgical lights 112 arrayed in a single housing 114 provide an illuminating light 118 toward illumination target 120 on table 124. The illumination target 120 is located by a device 116 which may be an ultrasonic detector. As FIG. 3 demonstrates, the prior art includes light housings 114 that are attached to a ceiling 122, thereby consuming overhead working space.

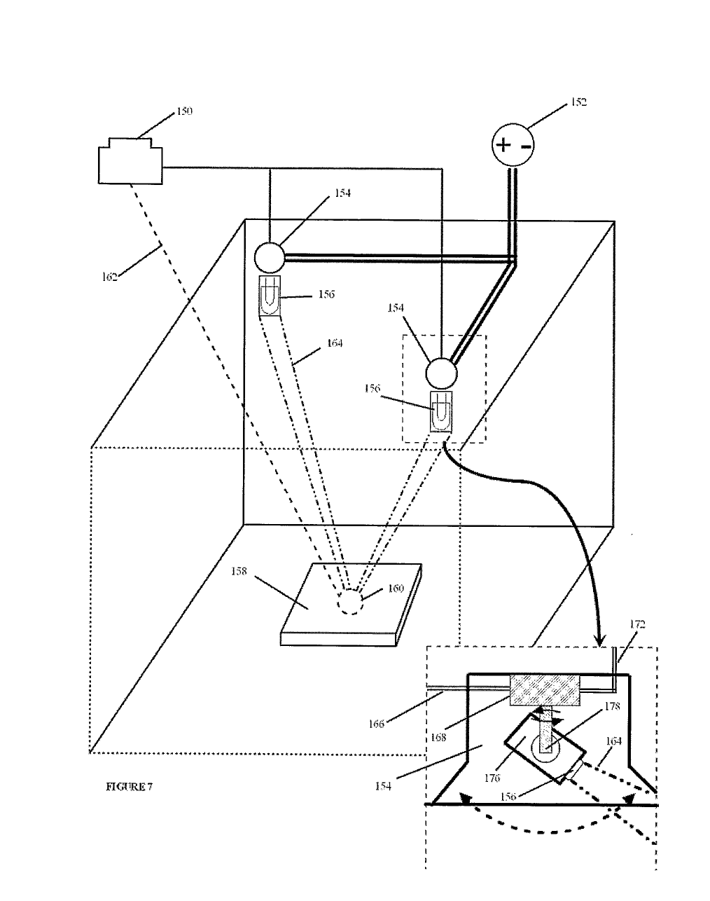

FIGS. 4, 5, and 6 demonstrate some of the deficiencies in the prior art. FIG. 4 shows two surgical lights 112 which are adjacent to each other and illuminating a target field 120 on table 124. The dotted arrow indicates the width of the illuminating beam 118. As FIG. 4 shows, if the illuminating source, namely surgical lights 112, is adjacent and near the target 120, they provide a focused and substantial illumination. However, as the target field is placed more distant from lights 112, as seen in FIG. 6, the beam width increases and hence becomes less focused. Light loses its intensity and focus with increasing distance from the source. In comparison, FIG. 5 shows lights 112 placed more distant from each other. Again, because the distance from source to target has been increased, the target is illuminated less well.

The present invention comprises numerous improvements over the prior art, as shown in FIG. 7. In FIG. 7, lights are housed independently of each other, thereby allowing very intense, focused lights to be used. The use of very intense, focused lights compensate for the effect (light intensity and focus diminishment) demonstrated in FIG. 5. Each light source, such as a lamp, must provide about at least 100,000 Lux with a color temperature approximately around 5600 K. The lamp should be of a metal-halide type. As such, the lights are freed from the constraint of being housing together to provide the necessary intensity and focus as in the prior art shown in FIGS. 2 and 3. Further, individual housing of lights allows each light to be housed within a recessed cavity within the ceiling, thereby freeing valuable overhead working space.

The recessed housing 154, in one embodiment, is detailed in the lower right of FIG. 7. Shown is lamp 156 contained within lamp housing 176 which may be attached to a powered swiveling means and support 178, actuated by control box 150. The swiveling means would allow directional travel of the lamp housing 176 and its lamp 156 along the path described by the bidirectional dotted arrow. Support 178 itself may be attached to a mount and swivel means 168 which would allow directional travel of the lamp housing 176 and its lamp 156 along the path described by the two oppositely orientated solid arrows. Power is provided by cabling 172, which is supplied from an outside AC or DC source 152. Further, control signals are provided from control box 150 from cabling 166. The control signal actuates 170 and 176 as necessary by computing the appropriate positioning of lamp 156 based on a distance signal 162 (sent wirelessly in the RF band) provided by locator 160 on table 158.

In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention. It is further known that other modifications may be made to the present invention, without departing the scope of the invention, as noted in the appended claims.

I claim:

1. A hospital operating room illumination apparatus comprising
at least one housing recessed into a ceiling of an operating room;
a control box which controls at least one powered actuator;
a locator adapted to transmit a distance signal,
substantially in the radio-frequency electromagnetic spectrum,
representative of the actual distance between said housing and said locator;
a communications device on said control box which receives said distance signal wirelessly from said locator;
at least one light in any said housing with no other lights in said housing,
adapted to provide a focused beam of light of substantially at least 100,000 Lux, with a color temperature around 5600 K, and with said light of a metal-halide type, and
adapted, when used with at least one other light, to provide a light source directed at a point from at least two spatially separate regions.
a positioning system in said control box which includes a servo control circuit adapted to receive a distance signal from said locator device and adapted to interpret said distance signal and then activate any said actuators coupled to any said housing, thereby adjusting at least one powered swivel, joint, castor, or bushing to illuminate said locator device by reference to said distance signal;

2. The hospital operating room illumination apparatus of claim 1 in which the number of lights is at least two and said lights are arrayed about a central point.

3. The hospital operating room illumination apparatus of claim 1 in which said locator is a Bluetooth device.

4. The hospital operating room illumination apparatus of claim 1 in which said locator is composed of biocompatible materials.

5. The hospital operating room illumination apparatus of claim 1 in which said locator is sterilizable.

6. The hospital operating room illumination apparatus of claim 1 in which said locator is disposable and interchangeable with like locators.

7. A method of using a means for illuminating a point in an operating room, comprising:
providing an operating room light as in claim 1;
placing said locator on a point to be illuminated;
allowing said locator to communicate with said control box;
allowing said control box to activate the servo control circuit in response to communication by activating said actuator;
allowing said actuator to use the position means, such as said swivel, joint, castor, or bushing, to position said light such that said light illuminates said point to be illuminated.

8. The hospital operating room illumination method of claim 7 in which the number of lights is at least two and said lights are arrayed about a central point.

9. The hospital operating room illumination method of claim 7 in which said locator is a Bluetooth device.

10. The hospital operating room illumination method of claim 7 in which said locator is composed of biocompatible materials.

11. The hospital operating room illumination method of claim 7 in which said locator is sterilizable.

12. The hospital operating room illumination method of claim 7 in which said locator is disposable and interchangeable with like locators.
